(19) United States
(12) Patent Application Publication
(10) Pub. No.: US 2022/0064280 A1
(43) Pub. Date: Mar. 3, 2022

(54) METHODS OF TREATING PSORIASIS

(71) Applicant: ELI LILLY AND COMPANY, INDIANAPOLIS, IN (US)

(72) Inventors: STUART WILLIAM FRIEDRICH, RONDEAU, ONTARIO (CA); Paul Alan Klekotka, San Diego, CA (US); Jay Lawrence Tuttle, San Marcos, CA (US)

(21) Appl. No.: 17/275,027

(22) PCT Filed: Sep. 5, 2019
(86) PCT No.: PCT/US2019/049648

§ 371 Date: Mar. 10, 2021

Related U.S. Application Data
(60) Provisional application No. 62/729,435, filed on Sep. 11, 2018.

(51) Int. Cl. C07K 16/24; A61P 17/06

(52) U.S. Cl. CPC C07K 16/244; A61K 2039/545; A61P 17/06

(57) ABSTRACT

The present invention generally relates to the treatment of psoriasis with an antibody that binds to the p19 subunit of human IL-23, in particular dosage regimens for the treatment of the disease.

This invention generally relates to method of treating inflammatory diseases, for example, psoriasis, with antibodies that bind to the p19 subunit of human IL-23.

Psoriasis is a chronic, immune-mediated, inflammatory skin disease, with a global incidence of approximately 2%, associated with significant morbidity and can have a substantial psychosocial impact on quality of life and well-being of patients. Plaque psoriasis is the most common form and affects approximately 80-90% of patients, manifesting as raised plaques on the skin; the disease usually begins in late adolescence and early adulthood and may persist through adult life. The extent of the affected body surface area (BSA) and the degree of skin manifestations, including erythema, induration, and scaling, defines the severity of psoriasis with approximately 20-30% of patients having moderate-to-severe disease.

Histologically, psoriasis is characterized by inflammatory infiltrate and hyper-proliferative keratinocytes, which retain intact nuclei (parakeratosis), elongation of rete ridges, and hyper-convoluted vasculature in the papillary dermis. The infiltrate consists of prominent T cells, dendritic cells (DCs), and neutrophils in the dermis. The dysregulation of the immune system, especially the activation of pathogenic T cells, has been well demonstrated to play an important role in psoriasis development.

A typical organ-specific T-cell-driven inflammatory disease, psoriasis had been considered a T helper (Th) 1-type skin disease for decades until a new Th population, Th17, was identified (Steinman L, Nat Med., 13(2), pp 139-145, 2007). Substantial clinical and laboratory research observations revealed that the interleukin (IL)-23/Th17 axis is essential in the pathogenesis of psoriasis (Di Cesare et al., J Invest Dermatol., 129(6), pp 1339-1350, 2009). IL-23, a member of the IL-12 family of cytokines, is a heterodimeric protein comprised of two subunits; the p40 subunit, which it shares with IL-12, and the p19 subunit, believed to be specific to IL-23. IL-23 is produced by antigen-presenting cells, such as DCs and macrophages, and plays an important role in maintenance and amplification of Th17 cells (Lee et al., J Exp Med., 199(1), pp 125-130 2004). In addition, Th17 cells and their downstream effector molecules, including IL-17A, IL-17F, IL-21, IL-22, and tumor necrosis factor alpha (TNF-α), are found at increased levels in human psoriatic skin lesions and circulation (Boniface et al., Clin Exp Immunol., 150(3), pp 407-415, 2007; Kagami et al., J Invest Dermatol., 130(5), pp 1373-1383, 2010).

Treatment of psoriasis with biologic therapy, particularly with those agents targeting the IL-23/Th17 axis, has demonstrated clinical activity in patients with psoriasis (Crow J M, Nature, 492(7429), S58-S59, 2012). Agents specifically targeting the IL-23 p19 subunit have demonstrated clinical activity in psoriasis (Kopp et al., Nature, 14175, 2015).

There is a need for treatment options for psoriasis that lead to favourable outcomes for patients, for example in terms of efficacy, safety and/or tolerability of the treatment.

The present invention addresses the above needs and provides methods for treating inflammatory diseases, in particular methods comprising administering an anti-IL-23p19 antibody to a patient in certain amounts and/or at certain intervals. In one aspect, the present invention provides a method for the treatment of psoriasis comprising administering mirikizumab to a patient, said method comprising:
a) administering at least one induction dose of mirikizumab to the patient, wherein the induction dose comprises 20 mg to 600 mg of mirikizumab; and
b) administering at least one maintenance dose of mirikizumab to the patient after the last induction dose is administered, wherein the maintenance dose comprises 20 mg to 600 mg of mirikizumab.

In an embodiment of the present invention, the psoriasis is moderate to severe plaque psoriasis.

In a further embodiment of the present invention, the psoriasis is scalp psoriasis.

In a further embodiment of the present invention, the patient is biologic-naïve. In an alternative embodiment of the method of the present invention, the patient is biologic-experienced.

In a still further embodiment of the present invention, the at least one induction dose comprises 20 mg, 30 mg, 60 mg, 100 mg, 120 mg, 125 mg, 250 mg, 300 mg, 350 mg, 400 mg or 600 mg of mirikizumab.

Preferably, the at least one induction dose comprises 250 mg of mirikizumab.

In a still further embodiment of the present invention, one, two, three or four induction doses are administered to the patient.

Preferably, two induction doses are administered to the patient at 8-week intervals.

Alternatively preferably, three induction doses are administered to the patient at 4-week intervals.

Further alternatively preferably, four induction doses are administered to the patient at 4-week intervals.

In a still further embodiment of the present invention, the at least one induction dose is administered subcutaneously.

In a still further embodiment of the present invention, the at least one maintenance dose comprises 20 mg, 30 mg, 100 mg, 120 mg, 125 mg, 250 mg, 300 mg, 350 mg, 400 mg or 600 mg of mirikizumab.

Preferably, the at least one maintenance dose comprises 125 mg or 250 mg of mirikizumab.

In a still further embodiment of the present invention, the at least one maintenance dose is administered 2-16 weeks after the last induction dose is administered.

In a still further embodiment of the present invention, the at least one maintenance dose is administered 2 weeks, 3 weeks, 4 weeks, 5 weeks, 6 weeks, 7 weeks, 8 weeks, 12 weeks or 16 weeks after the last induction dose is administered.

Preferably, the at least one maintenance dose is administered 4 weeks after the last induction dose is administered.

Alternatively preferably, the at least one maintenance dose is administered 8 weeks after the last induction dose is administered.

Further alternatively preferably, the at least one maintenance dose is administered 12 weeks after the last induction dose is administered.

Still further alternatively preferably, the at least one maintenance dose is administered 16 weeks after the last induction dose is administered.

In a still further embodiment of the present invention, multiple maintenance doses are administered to a patient and wherein the first maintenance dose is administered 2 to 16 weeks after the last induction dose is administered.

In a still further embodiment of the present invention, the first maintenance dose is administered 2 weeks, 3 weeks, 4 weeks, 5 weeks, 6 weeks, 7 weeks, 8 weeks, 12 weeks or 16 weeks after the last induction dose is administered.

Preferably, the first maintenance dose is administered 4 weeks after the last induction dose is administered.

Alternatively preferably, the first maintenance dose is administered 8 weeks after the last induction dose is administered.

Further alternatively preferably, the first maintenance dose is administered 12 weeks after the last induction dose is administered.

Still further alternatively preferably, the first maintenance dose is administered 16 weeks after the last induction dose is administered.

In a still further embodiment of the present invention, one or more further maintenance dose(s) are administered at 4, 8 or 12-week interval(s) after administration of the first maintenance dose.

Preferably, one or more further maintenance dose(s) are administered at 4-week interval(s).

Alternatively preferably, one or more further maintenance dose(s) are administered at 8-week interval(s).

Further alternatively preferably, one or more further maintenance dose(s) are administered at 12-week interval(s).

In a still further embodiment of the present invention, the maintenance dose(s) are administered by subcutaneous injection.

In a preferred embodiment of the present invention, the method of treating psoriasis comprises:
a) administering (i) two, three or four induction doses of mirikizumab to the patient by subcutaneous injection, wherein each induction dose comprises 250 mg of mirikizumab; and
b) administering at least one maintenance dose of mirikizumab to the patient by subcutaneous injection at 4 week or 8 week intervals, wherein the first maintenance dose is administered 4 weeks or 8 weeks after the last induction dose is administered and wherein each maintenance dose comprises 125 mg or 250 mg of mirikizumab,

wherein the psoriasis is moderate to severe plaque psoriasis.

Preferably, two induction doses of mirikizumab are administered at 8-week intervals and the first maintenance dose is administered 8 weeks after the last induction dose is administered.

Alternatively preferably, three induction doses of mirikizumab are administered at 4-week intervals and the first maintenance dose is administered 4 weeks after the last induction dose is administered.

Further alternatively preferably, four induction doses of mirikizumab are administered at 4 week intervals and the first maintenance dose is administered 4 weeks after the last induction dose is administered.

Further preferably, each maintenance dose comprises 250 mg of mirikizumab.

Alternatively preferably, each maintenance dose comprises 125 mg of mirikizumab.

In another aspect, the present invention provides a method of treating psoriasis comprising administering mirikizumab to a patient, said method comprising:
a) administering one or more induction dose(s) of mirikizumab to the patient during an induction period, wherein the one or more induction dose(s) each comprise 20 mg to 600 mg of mirikizumab;
b) determining the disease activity level of the patient at the end of the induction period and
   i) administering one or more maintenance dose(s) to a patient that has not achieved a high level of clinical response at the end of the induction period, wherein the one or more maintenance dose(s) each comprise 20 mg to 600 mg of mirikizumab; and
   ii) continuing assessment of the disease activity level of a patient that has achieved a high level of clinical response beyond the induction period and administering one or more maintenance dose(s) to the patient if the patient's disease activity level falls below a high level of clinical response, wherein the one or more maintenance dose(s) are administered until the patient re-achieves a high level of clinical response, and wherein the one or more maintenance dose(s) each comprise 20 mg to 600 mg of mirikizumab.

In one embodiment of the present invention, a high level of clinical response is a disease activity level of ≥PASI 90 or ≥sPGA (0, 1).

This treatment regimen enables patients that have not achieved a high level of clinical response at the end of the induction period to continue treatment with one or more maintenance doses in order to continue progression toward a high level of clinical response. Those patients that have achieved a high level of clinical response at the end of the induction period are treated as needed (PRN). That is, the patient is treated with one or more maintenance dose(s) if the patient's disease activity level falls below a high level of clinical response until the patient re-achieves a high level of clinical response.

In a further embodiment of the present invention, the psoriasis is moderate to severe plaque psoriasis.

In a still further embodiment of the present invention, the psoriasis is scalp psoriasis.

In a still further embodiment of the present invention, the patient is biologic-naïve. In an alternative embodiment of the present invention, the patient is biologic-experienced.

In a still further embodiment of the present invention, the one or more induction dose(s) each comprise 20 mg, 30 mg, 100 mg, 120 mg, 125 mg, 250 mg, 300 mg, 350 mg, 400 mg or 600 mg of mirikizumab.

Preferably, the one more induction dose(s) each comprise 250 mg of mirikizumab.

In a still further embodiment of the present invention, one, two, three or four induction doses are administered to the patient.

In a still further embodiment of the present invention, the induction period is 12 weeks or 16 weeks.

Preferably, the induction period is 16 weeks and two induction doses are administered to the patient at 8-week intervals.

Alternatively preferably, the induction period is 12 weeks and three induction doses are administered to the patient at 4-week intervals.

Further alternatively, the induction period is 16 weeks and four induction doses are administered to the patient at 4-week intervals.

In a still further embodiment of the present invention, the at least one induction dose is administered subcutaneously.

In a still further embodiment of the present invention, the one or more maintenance dose(s) each comprise 20 mg, 30 mg, 100 mg, 120 mg, 125 mg, 250 mg, 300 mg, 350 mg, 400 mg or 600 mg of mirikizumab.

Preferably, the one or more maintenance dose(s) each comprise 125 mg or 250 mg of mirikizumab.

In a still further embodiment of the present invention, the one or more maintenance dose(s) are administered by subcutaneous injection.

In a still further embodiment of the present invention, if the patient has not achieved a high level of clinical response at the end of the induction period, a first maintenance dose is administered 2 to 16 weeks after the last induction dose is administered.

In a still further embodiment of the present invention, if the patient has not achieved a high level of clinical response at the end of the induction period, the first maintenance dose is administered 2 weeks, 3 weeks, 4 weeks, 5 weeks, 6 weeks, 7 weeks, 8 weeks, 12 weeks or 16 weeks after the last induction dose is administered.

Preferably, the first maintenance dose is administered 4 weeks after the last induction dose is administered.

Alternatively preferably, wherein the first maintenance dose is administered 8 weeks after the last induction dose is administered.

Further alternatively preferably, the first maintenance dose is administered 12 weeks after the last induction dose is administered.

Still further alternatively preferably, the first maintenance dose is administered 12 weeks after the last induction dose is administered.

In a still further embodiment of the present invention, one or more further maintenance dose(s) are administered at 4, 8 or 12 week interval(s) after administration of the first maintenance dose.

Preferably, one or more further maintenance dose(s) are administered at 4 week interval(s).

Alternatively preferably, one or more further maintenance dose(s) are administered at 8 week interval(s).

In a still further embodiment of the present invention, if the patient has achieved a high level of clinical response at the end of the induction period and the patient's disease activity level has subsequently fallen below a high level of clinical response:
i) a first maintenance dose is administered to the patient;
ii) the disease activity level is assessed at 4-week, 8-week or 12 week interval(s) after administration of the first maintenance dose; and
iii) a further maintenance dose is administered after each assessment of the disease activity level if the patient has not achieved a high level of clinical response and until the patient re-achieves a high level of clinical response.

Those patients that have achieved a high level of clinical response at the end of the induction period are treated as needed (PRN). If the patient's disease activity level falls below a high level of clinical response, the patient is administered a first maintenance dose. The patient's disease activity level is assessed 4 weeks (or alternatively, 8 weeks or 12 weeks) after administration of the first maintenance dose. If the patient has not re-achieved a high level of clinical response after administration of the first maintenance dose, a further maintenance dose is administered. This assessment/treatment cycle continues until the patient re-achieves a high level of clinical response. Thereafter, the patient is again treated as needed, i.e. treatment with further maintenance dose(s) is suspended until the disease level of the patient falls below a high level of clinical response again.

In a still further embodiment of the present invention, the disease activity is assessed at 4-week intervals after administration of the first maintenance dose and a further maintenance dose is administered after each assessment until the patient re-achieves a high level of clinical response.

In a still further alternative embodiment of the present invention, the disease activity is assessed at 8-week intervals after administration of the first maintenance dose and a further maintenance dose is administered after each assessment until the patient re-achieves a high level of clinical response.

In a still further embodiment of the present invention, the one or more maintenance dose(s) are administered by subcutaneous injection.

In a further aspect, the present invention provides a method of treating psoriasis comprising administering mirikizumab to a patient, said method comprising:
i) administering one or more induction dose(s) of mirikizumab until the patient achieves clinical remission, wherein the one or more induction dose(s) each comprise 20 mg to 600 mg of mirikizumab; and
ii) monitoring the disease activity level of the patient and administering one or more maintenance dose(s) of mirikizumab if the disease activity of the patient falls below clinical remission, wherein the one or more maintenance dose(s) are administered until the patient re-achieves clinical remission, and wherein the one or more maintenance dose(s) each comprise 20 mg to 600 mg of mirikizumab.

In an embodiment of the present invention, clinical remission is a disease activity level of PASI 100 or sPGA (0).

This treatment regimen involves treatment of a patient until he/she has achieved clinical remission and thereafter treating the patient as needed (PRN).

In a further embodiment of the present invention, the psoriasis is moderate to severe plaque psoriasis.

In a still further embodiment of the present invention, the patient is biologic-naïve. In an alternative embodiment, the patient is biologic-experienced.

In a still further embodiment of the present invention, the disease activity is assessed at 4-week, 8-week or 12 week interval(s) after administration of the first induction dose and further induction dose(s) are administered after assessment of the disease activity level if the patient has not achieved clinical remission.

The patient's disease activity level is assessed 4 weeks (or alternatively, 8 weeks or 12 weeks) after administration of the first induction dose. If the patient has not achieved clinical remission after administration of the first induction dose, a further induction dose is administered. This assessment/treatment cycle continues until the patient achieves clinical remission.

In a still further embodiment of the present invention, the one or more induction dose(s) each comprise 20 mg, 30 mg, 100 mg, 120 mg, 125 mg, 250 mg, 300 mg, 350 mg, 400 mg or 600 mg of mirikizumab.

Preferably, the one or more induction dose(s) each comprise 250 mg of mirikizumab.

In a still further embodiment of the present invention, if the disease activity level of the patient falls below clinical remission:
i) a first maintenance dose of mirikizumab is administered to the patient;
ii) disease activity is assessed at 4-week, 8-week or 12 week interval(s) after administration of the first maintenance dose; and
iii) further maintenance dose(s) are administered after each assessment of the disease activity level if the patient has not re-achieved clinical remission.

If the patient's disease activity level falls below a clinical remission, the patient is administered a first maintenance dose. The patient's disease activity level is assessed 4 weeks (or alternatively, 8 weeks or 12 weeks) after administration of the first maintenance dose. If the patient has not re-achieved clinical remission after administration of the first maintenance dose, a further maintenance dose is administered. This assessment/treatment cycle continues until the patient re-achieves clinical remission. Thereafter, the patient is again treated as needed, i.e. treatment with further maintenance dose(s) is suspended until the disease level of the patient falls below clinical remission again.

In a still further embodiment of the present invention, the one or more maintenance dose(s) each comprise 20 mg, 30 mg, 100 mg, 120 mg, 125 mg, 250 mg, 300 mg, 350 mg, 400 mg or 600 mg of mirikizumab.

Preferably, the one or more maintenance dose(s) each comprise 125 mg or 250 mg of mirikizumab.

The methods of the present invention provide the advantage of enabling patients to experience clinical improvement while receiving fewer administrations of the mirikizumab.

In a further aspect, the present invention provides mirikizumab for use in the treatment of psoriasis, wherein the treatment comprises:
a) administering at least one induction dose of mirikizumab, wherein the induction dose comprises 20 mg to 600 mg of mirikizumab; and
b) administering at least one maintenance dose of mirikizumab after the last induction dose is administered, wherein the maintenance dose comprises 20 mg to 600 mg of mirikizumab.

In an embodiment of the present invention, the psoriasis is moderate to severe plaque psoriasis.

In a further embodiment of the present invention, the psoriasis is scalp psoriasis.

In a still further embodiment of the present invention, the patient is biologic-naïve. In an alternative embodiment of the method of the present invention, the patient is biologic-experienced.

In a still further embodiment of the present invention, the at least one induction dose comprises 20 mg, 30 mg, 100 mg, 120 mg, 250 mg, 300 mg, 350 mg, 400 mg or 600 mg of mirikizumab.

Preferably, the at least one induction dose comprises 250 mg of mirikizumab.

In a still further embodiment of the present invention, one, two, three or four induction doses are administered to the patient.

Preferably, two induction doses are administered to the patient at 8-week intervals.

Alternatively preferably, three induction doses are administered to the patient at 4-week intervals.

Alternatively preferably, four induction doses are administered to the patient at 4-week intervals.

In a still further embodiment of the present invention, the at least one induction dose is administered subcutaneously.

In a still further embodiment of the present invention, the at least one maintenance dose comprises 20 mg, 30 mg, 100 mg, 120 mg, 125 mg, 250 mg, 300 mg, 350 mg, 400 mg or 600 mg of mirikizumab.

Preferably, the at least one maintenance dose comprises 125 mg or 250 mg of mirikizumab.

In a still further embodiment of the present invention, the at least one maintenance dose is administered 2-16 weeks after the last induction dose is administered.

In a still further embodiment of the present invention, the at least one maintenance dose is administered 2 weeks, 3 weeks, 4 weeks, 5 weeks, 6 weeks, 7 weeks, 8 weeks, 12 weeks or 16 weeks after the last induction dose is administered.

Preferably, the at least one maintenance dose is administered 4 weeks after the last induction dose is administered.

Alternatively preferably, the at least one maintenance dose is administered 8 weeks after the last induction dose is administered.

Further alternatively preferably, the at least one maintenance dose is administered 12 weeks after the last induction dose is administered.

Still further alternatively preferably, the at least one maintenance dose is administered 16 weeks after the last induction dose is administered.

In a still further embodiment of the present invention, multiple maintenance doses are administered to a patient and wherein the first maintenance dose is administered 2 to 16 weeks after the last induction dose is administered.

In a still further embodiment of the present invention, the first maintenance dose is administered 2 weeks, 3 weeks, 4 weeks, 5 weeks, 6 weeks, 7 weeks, 8 weeks, 12 weeks or 16 weeks after the last induction dose is administered.

Preferably, the first maintenance dose is administered 4 weeks after the last induction dose is administered.

Alternatively preferably, the first maintenance dose is administered 8 weeks after the last induction dose is administered.

Further alternatively preferably, the first maintenance dose is administered 12 weeks after the last induction dose is administered.

Still further alternatively preferably, the first maintenance dose is administered 16 weeks after the last induction dose is administered.

In a still further embodiment of the present invention, one or more further maintenance dose(s) are administered at 4, 8 or 12-week interval(s) after administration of the first maintenance dose.

Preferably, one or more further maintenance dose(s) are administered at 4-week interval(s).

Alternatively preferably, one or more further maintenance dose(s) are administered at 8-week interval(s).

Further alternatively preferably, one or more further maintenance dose(s) are administered at 12-week interval(s).

In a still further embodiment of the method of the present invention, the maintenance dose(s) are administered by subcutaneous injection.

In a preferred embodiment of the present invention, the treatment comprises:
a) administering (i) two, three or four induction doses of mirikizumab to the patient by subcutaneous injection, wherein each induction dose comprises 250 mg of mirikizumab; and
b) administering at least one maintenance dose of mirikizumab to the patient by subcutaneous injection at 4 week or 8 week intervals, wherein the first maintenance dose is administered 4 weeks or 8 weeks after the last induction dose is administered and wherein each maintenance dose comprises 125 mg or 250 mg of mirikizumab,

wherein the psoriasis is moderate to severe plaque psoriasis.

Preferably, two induction doses of mirikizumab are administered at 8-week intervals and the first maintenance dose is administered 8 weeks after the last induction dose is administered.

Alternatively preferably, three induction doses of mirikizumab are administered at 4-week intervals and the first maintenance dose is administered 4 weeks after the last induction dose is administered.

Further alternatively preferably, four induction doses of mirikizumab are administered at 4 week intervals and the first maintenance dose is administered 4 weeks after the last induction dose is administered.

Further preferably, each maintenance dose comprises 250 mg of mirikizumab.

Alternatively preferably, each maintenance dose comprises 125 mg of mirikizumab.

In an aspect of the present invention, mirikizumab is provided for use in the treatment of psoriasis, the treatment comprising:
a) administering one or more induction dose(s) of mirikizumab to a patient during an induction period, wherein the one or more induction dose(s) each comprise 20 mg to 600 mg of mirikizumab;
b) determining the disease activity level of the patient at the end of the induction period and
   i) administering one or more maintenance dose(s) to a patient that has not achieved a high level of clinical response at the end of the induction period, wherein the one or more maintenance dose(s) each comprise 20 mg to 600 mg of mirikizumab;
   ii) continuing assessment of the disease activity level of a patient that has achieved a high level of clinical response beyond the induction period and administering one or more maintenance dose(s) to the patient if the patient's disease activity level falls below a high level of clinical response, wherein the one or more maintenance dose(s) are administered until the patient re-achieves a high level of clinical response, and wherein the one or more maintenance dose(s) each comprise 20 mg to 600 mg of mirikizumab.

In one embodiment of the present invention, a high level of clinical response is a disease activity level of ≥PASI 90 or ≥sPGA (0, 1).

In a further embodiment of the present invention, the psoriasis is moderate to severe plaque psoriasis.

In a still further embodiment of the present invention, the psoriasis is scalp psoriasis.

In a still further embodiment of the present invention, the patient is biologic-naïve. In an alternative embodiment of the present invention, the patient is biologic-experienced.

In a still further embodiment of the present invention, the one or more induction dose(s) each comprise 20 mg, 30 mg, 100 mg, 120 mg, 125 mg, 250 mg, 300 mg, 350 mg, 400 mg or 600 mg of mirikizumab.

Preferably, the one or more induction dose(s) each comprise 250 mg of mirikizumab.

In a still further embodiment of the present invention, one, two, three or four induction dose(s) are administered to the patient.

In a still further embodiment of the present invention, the induction period is 12 weeks or 16 weeks.

Preferably, the induction period is 16 weeks and two induction doses are administered to the patient at 8-week intervals.

Alternatively preferably, the induction period is 12 weeks and three induction doses are administered to the patient at 4-week intervals.

Further alternatively, the induction period is 16 weeks and four induction doses are administered to the patient at 4-week intervals.

In a still further embodiment of the present invention, the one or more induction dose(s) are administered subcutaneously.

In a still further embodiment of the present invention, the one or more maintenance dose(s) each comprise 20 mg, 30 mg, 100 mg, 120 mg, 125 mg, 250 mg, 300 mg, 350 mg, 400 mg or 600 mg of mirikizumab.

Preferably, the one or more maintenance dose(s) each comprise 125 mg or 250 mg of mirikizumab.

In a still further embodiment of the present invention, the one or more maintenance dose(s) are administered by subcutaneous injection.

In a still further embodiment of the present invention, if the patient has not achieved a high level of clinical response at the end of the induction period, a first maintenance dose is administered 2 to 16 weeks after the last induction dose is administered.

In a still further embodiment of the present invention, if the patient has not achieved a high level of clinical response at the end of the induction period, a first maintenance dose is administered 2 weeks, 3 weeks, 4 weeks, 5 weeks, 6 weeks, 7 weeks, 8 weeks, 12 weeks or 16 weeks after the last induction dose is administered.

Preferably, the first maintenance dose is administered 4 weeks after the last induction dose is administered.

Alternatively preferably, wherein the first maintenance dose is administered 8 weeks after the last induction dose is administered.

Further alternatively preferably, the first maintenance dose is administered 12 weeks after the last induction dose is administered.

Still further alternatively preferably, the first maintenance dose is administered 12 weeks after the last induction dose is administered.

In a still further embodiment of the present invention, one or more further maintenance dose(s) are administered at 4, 8 or 12 week interval(s) after administration of the first maintenance dose.

Preferably, one or more further maintenance dose(s) are administered at 4 week interval(s).

Alternatively preferably, one or more further maintenance dose(s) are administered at 8 week interval(s).

In a still further embodiment of the present invention, if the patient has achieved a high level of clinical response at the end of the induction period and the patient's disease activity level has subsequently fallen below a high level of clinical response:
i) a first maintenance dose is administered to the patient;
ii) the disease activity level is assessed at 4-week, 8-week or 12 week interval(s) after administration of the first maintenance dose; and
iii) a further maintenance dose is administered after each assessment of the disease activity level if the patient has not achieved a high level of clinical response and until the patient re-achieves a high level of clinical response.

In a still further embodiment of the present invention, the disease activity is assessed at 4-week intervals after administration of the first maintenance dose and a further maintenance dose is administered after each assessment until the patient re-achieves a high level of clinical response.

In a still further alternative embodiment of the present invention, the disease activity is assessed at 8-week intervals after administration of the first maintenance dose and a further maintenance dose is administered after each assessment until the patient re-achieves a high level of clinical response.

In a still further embodiment of the present invention, the one or more maintenance dose(s) are administered by subcutaneous injection.

In a further aspect, the present invention provides mirikizumab for use in the treatment of psoriasis, the treatment comprising:
iv) administering one or more induction dose(s) of mirikizumab until the patient achieves clinical remission, wherein the one or more induction dose(s) each comprise 20 mg to 600 mg of mirikizumab; and
v) monitoring the disease activity level of the patient and administering one or more maintenance dose(s) of mirikizumab if the disease activity of the patient falls below clinical remission until the patient re-achieves clinical remission, wherein the one or more maintenance dose(s) each comprise 20 mg to 600 mg of mirikizumab.

In an embodiment of the present invention, clinical remission is a disease activity level of PASI 100 or sPGA (0).

In a further embodiment of the present invention, the psoriasis is moderate to severe plaque psoriasis.

In a still further embodiment of the present invention, the patient is biologic-naïve. In an alternative embodiment, the patient is biologic-experienced.

In a still further embodiment of the present invention, the disease activity is assessed at 4-week, 8-week or 12 week interval(s) after administration of the first induction dose and further induction dose(s) are administered after assessment of the disease activity level if the patient has not achieved clinical remission.

In a still further embodiment of the present invention, the one or more induction dose(s) each comprises 20 mg, 30 mg, 100 mg, 120 mg, 125 mg, 250 mg, 300 mg, 350 mg, 400 mg or 600 mg of mirikizumab.

Preferably, the one or more induction dose(s) each comprise 250 mg of mirikizumab.

In a still further embodiment of the present invention, if the disease activity level of the patient falls below clinical remission:
i) a first maintenance dose of mirikizumab is administered to the patient;
ii) disease activity is assessed at 4-week, 8-week or 12 week interval(s) after administration of the first maintenance dose; and
vi) further maintenance dose(s) are administered after each assessment of the disease activity level if the patient has not re-achieved clinical remission.

In a still further embodiment of the present invention, the one or more maintenance dose(s) each comprise 20 mg, 30 mg, 100 mg, 120 mg, 125 mg, 250 mg, 300 mg, 350 mg, 400 mg or 600 mg of mirikizumab.

Preferably, the one or more maintenance dose(s) comprises 125 mg or 250 mg of mirikizumab.

In a still further embodiment, the one or more maintenance dose(s) are administered by subcutaneous injection.

### FIGURES

FIG. 1 illustrates the percentage of PASI 90 responders for placebo subjects and subjects assigned to treatment with mirikizumab that have a <PASI 90 at Week 16 and are administered mirikizumab 300 mg SC Q8W during Weeks 16-52 of the maintenance period.

FIG. 2 illustrates the percentage of PASI 100 responders for placebo subjects and subjects assigned to treatment with mirikizumab that have a <PASI 90 at Week 16 and are administered mirikizumab 300 mg SC Q8W during Weeks 16-52 of the maintenance period.

FIGS. 3a, 3b and 3c illustrate the PASI 75, PASI 90 and PASI 1000 scores at Week 52 of exposure-naïve and prior-exposure patient groups with moderate-to-severe plaque psoriasis who did not achieve PASI 90 at Week 16

### DETAILED DESCRIPTION

Psoriasis is a chronic inflammatory disease of the skin characterized by dysfunctional keratinocyte differentiation and hyper-proliferation and marked accumulation of inflammatory T cells and dendritic cells. For example, the immunological disease includes plaque psoriasis, for example chronic plaque psoriasis, for example moderate to severe chronic plaque psoriasis, for example in patients who are candidates for systemic therapy or phototherapy.

There are various measurements of disease activity level.

For psoriasis, disease severity can be characterized by body surface area (BSA) involvement with <5% being considered mild, 5-10% moderate and >10% severe. Percent BSA is evaluated as the percent involvement of psoriasis on each patient's BSA on a continuous scale from 0% (no involvement) to 100% (full involvement), where 1% corresponds to the size of the patient's hand (including the palm, fingers, and thumb) (National Psoriasis Foundation 2009).

In some cases, disease status is measured using the Psoriasis Area and Severity Index (PASI). The PASI is an accepted primary efficacy measurement for this phase of development of psoriasis treatments. The PASI combines assessments of the extent of body-surface involvement in four anatomical regions (head, trunk, arms, and legs) and the severity of scaling, redness, and plaque induration/infiltration (thickness) in each region, yielding an overall score of 0 for no psoriasis to 72 for the most severe disease (Fredriksson and Pettersson, Dermatologica, 157(4), pp 238-244, 1978). The PASI has been the most frequently used endpoint and measure of psoriasis severity in clinical trials (Menter et al., J Am Acad Dermatol., 58(5), pp 826-850, 2008). A clinically meaningful response is a PASI 75, which represents at least a 75% decrease (improvement) from the baseline PASI score. Higher levels of clearance (PASI 90), as well as complete resolution of psoriasis (PASI 100), have become additional endpoints because of the increasing recognition of the association of higher clearance with greater health-related quality of life (HRQoL). The percentage of patients reaching PASI₇₅ (PASI 75), a 75% reduction in score from baseline at a certain time (for example, at week 12 or week 16), may be used as a primary endpoint in psoriasis treatment, for example in psoriasis treatment trials. Alternatively, the percentage of patients reaching a PASI₉₀ (PASI 90), a 90% reduction in score from baseline at a certain time (for example, at week 12 or week 16) is used as primary endpoint in psoriasis treatment, for example in psoriasis treatment trials. Further alternatively, the percentage of patients reaching a PASI₁₀₀ (PASI 100), a 100% reduction in score from baseline at a certain time (for example, at week 12 or week 16) is used as primary endpoint in psoriasis treatment, for example in psoriasis treatment trials.

In some cases, disease status is measured using the Static Physician's Global Assessment (SPGA). The sPGA is the physician's global assessment of the patient's psoriasis lesions at a given time point (EMA 2004). Plaques are assessed for induration, erythema, and scaling as shown in Table 1.

TABLE 1
Static Physician Global Assessment (sPGA) Scale

| Score | Category | Category Description |
|---|---|---|
| 0 | Clear | Plaque elevation = 0 (no elevation over normal |
| | | skin) |
| | | Scaling = 0 (no scale) |
| | | Erythema = 0 (residual post-inflammatory |
| | | hyperpigmentation or hypopigmentation may be |
| | | present) |
| 1 | Minimal | Plaque elevation = ± (possible but difficult to |
| | | ascertain whether there is a slight elevation above |
| | | normal skin) |
| | | Scaling = ± (surface dryness with some white |
| | | coloration) |
| | | Erythema = up to moderate (up to definite red |
| | | coloration) |
| 2 | Mild | Plaque elevation = slight (slight but definite |
| | | elevation, typically edges are indistinct or sloped) |
| | | Scaling = fine (fine scale partially or mostly |
| | | covering lesions) |
| | | Erythema = up to moderate (up to definite red |
| | | coloration) |
| 3 | Moderate | Plaque elevation = moderate (moderate elevation |
| | | with rough or sloped edges) |
| | | Scaling = coarser (coarse scale covering most of all |
| | | of the lesions) |
| | | Erythema = moderate (definite red coloration) |
| 4 | Severe | Plaque elevation = marked (marked elevation |
| | | typically with hard or sharp edges) |
| | | Scaling = coarse (coarse, non-tenacious scale |
| | | predominates covering most or all of the lesions) |
| | | Erythema = severe (very bright red coloration) |
| 5 | Very Severe | Plaque elevation = very marked (very marked |
| | | elevation typically with hard sharp edges) |
| | | Scaling = very coarse (coarse, thick tenacious scale |
| | | over most of all the lesions; rough surface) |
| | | Erythema = very severe (extreme red coloration; |
| | | dusky to deep red coloration) |

For the analysis of responder rates, the sPGA scores are rounded to the nearest whole number, and the patient's psoriasis is assessed as clear (0), minimal (1), mild (2), moderate (3), severe (4), or very severe (5).

The Itch NRS is a patient-administered, 11-point horizontal scale anchored at 0 and 10, with 0 representing “no itch” and 10 representing “worst itch imaginable.” Overall severity of a patient's itching from psoriasis is indicated by circling the number that best describes the worst level of itching in the past 24 hours.

The Nail Psoriasis Severity Index (NAPSI) is used to evaluate the severity of fingernail bed psoriasis and fingernail matrix psoriasis by area of involvement in the fingernail unit. In this study, only fingernail involvement will be assessed. The fingernail is divided with imaginary horizontal and longitudinal lines into quadrants. Each fingernail is given a score for fingernail bed psoriasis (0 to 4) and fingernail matrix psoriasis (0 to 4) depending on the presence (score of 1) or absence (score of 0) of any of the features of fingernail bed and fingernail matrix psoriasis in each quadrant. The NAPSI score of a fingernail is the sum of scores in fingernail bed and fingernail matrix from each quadrant (maximum of 8). Each fingernail is evaluated, and the sum of all the fingernails is the total NAPSI score (range, 0 to 80).

The Psoriasis Scalp Severity Index (PSSI) measures the affected scalp area and the severity of clinical symptoms. The PSSI is a composite score derived from the sum of scores for erythema, induration, and desquamation multiplied by a score for the extent of scalp area involved (range, 0 to 72). Higher scores indicate worse severity (Thaçi et al., J Eur Acad Dermatol Venerol., 29(2), pp 353-360, 2015).

The Palmoplantar Psoriasis Severity Index (PPASI) is a composite score derived from the sum of scores for erythema, induration, and desquamation multiplied by a score for the extent of palm and sole area involvement (range, 0 to 72).

The Dermatology Life Quality Index (DLQI) is a validated, dermatology-specific, patient-reported measure that evaluates a patient's HRQoL. This questionnaire has 10 items that are grouped in 6 domains, namely symptoms and feelings, daily activities, leisure, work and school, personal relationships, and treatment. The recall period of this scale is over the “last week”. Response categories include “not at all,” “a little,” “a lot,” and “very much,” with corresponding scores of 0, 1, 2, and 3, respectively, and unanswered (“not relevant”) responses scored as “0”. Totals range from 0 to 30 (less to more impairment) (Finlay and Khan, Clin Exp Dermatol., 19(3), pp 210-216, 1994; Basra et al., Br J Dermatol., 159(5), pp 997-1035, 2008). A DLQI total score of 0 to 1 is considered as having no effect on a patient's HRQoL, and a 5-point change from baseline is considered as the minimal clinically important difference (MCID) threshold (Khilji et al., Br J Dermatol., 147 (supplement 62), 50, 2002; Hongbo et al., J Invest Dermatol., 125(4), pp 659-664, 2005).

The Psoriasis Symptoms Scale (PSS) is a patient-administered assessment of four symptoms (itch, pain, stinging, and burning); 3 signs (redness, scaling, and cracking); and one item on the discomfort related to symptoms/signs. Respondents are asked to answer the questions based on their psoriasis symptoms. The overall severity for each individual symptom/sign from the patient's psoriasis is indicated by selecting the number from a numeric rating scale (NRS) of 0 to 10 that best describes the worst level of each symptom/sign in the past 24 hours, where 0 is no symptom/sign and 10 is worst imaginable symptom/sign. The symptom severity scores, ranging from 0 to 10, are the values of the selected numbers indicated by the patient on the instrument's horizontal scale. Each of the 8 individual items will receive a score of 0 to 10 and will be reported as item scores for itch, pain, stinging, burning, redness, scaling, cracking, and discomfort. In addition, a symptoms score ranging from 0 (no symptoms) to 40 (worst imaginable symptoms) and a signs score of 0 (no signs) to 30 (worst imaginable signs) will be reported.

The Patient's Global Assessment of Psoriasis (PatGA) is a patient-reported, single-item scale on which patients are asked to rank, by selecting a number on a 0 to 5 NRS, the severity of their psoriasis “today,” from 0 (clear/no psoriasis) to 5 (severe).

As used herein, the terms “treating,” “treat,” or “treatment,” refer to restraining, slowing, lessening, reducing, or reversing the progression or severity of an existing symptom, disorder, condition, or disease, or ameliorating clinical symptoms and/or signs of a condition. Beneficial or desired clinical results include, but are not limited to, alleviation of symptoms, diminishment of the extent of a disease or disorder, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable. Those in need of treatment include those already with the disease.

As used herein, “clinical remission” means achievement of a disease activity level of PASI 100, sPGA (0), or equivalent thereof in other measurements of psoriasis disease activity level.

As used herein, “clinically meaningful response” means achievement of a disease activity level of PASI 75, sPGA (2), or equivalent thereof in other measurements of psoriasis disease activity level.

As used herein, “high level of clinical response” means achievement of a disease activity level of PASI 90, sPGA (0,1), or equivalent thereof in other measurements of psoriasis disease activity level.

As used herein, “induction period” refers to a period of treatment of a patient comprising administration of an antibody that binds to the p19 subunit of human IL-23, in particular, mirikizumab, to the patient in order to achieve a desired therapeutic effect or achieve progression toward a desired therapeutic effect, the desired therapeutic effect being induction of clinical remission (as defined hereinabove) and/or a clinically meaningful response (as defined hereinabove), and/or a high level of clinical response (as defined hereinabove). The “induction period” may be 4, 8, 12 or 16 weeks in duration.

As used herein, “induction dose” refers to a first dose of an antibody that binds to the p19 subunit of human IL-23, in particular, mirikizumab, administered to a patient in order to achieve a desired therapeutic effect or achieve progression toward a desired therapeutic effect, the desired therapeutic effect being induction of clinical remission (as defined hereinabove) and/or a clinically meaningful response ((as defined hereinabove) and/or a high level of clinical response (as defined hereinabove). The “induction dose” can be a single dose or, alternatively, a set of doses. The “induction dose” is administered during the induction period.

As used herein, “maintenance period” refers to refers to a period of treatment comprising administration of an antibody that binds to the p19 subunit of human IL-23, in particular, mirikizumab, to a patient in order to maintain a desired therapeutic effect and/or continue progression towards achievement of a desired therapeutic effect, the desired therapeutic effect being clinical remission (as defined hereinabove and/or a clinically meaningful response (as defined hereinabove), and/or a high level of clinical response ((as defined hereinabove). The “maintenance period” follows the induction period, and, therefore, is initiated once a desired therapeutic effect and/or progression towards achievement of a desired therapeutic effect is achieved.

As used herein, “maintenance dose” refers to a subsequent dose of an antibody that binds to the p19 subunit of human IL-23, in particular, mirikizumab, administered to a patient to maintain or continue progression toward a desired therapeutic effect, namely, clinical remission (as defined hereinabove) and/or a clinically meaningful response and/or a high level of clinical response (as defined hereinabove). A “maintenance dose” is administered subsequent to the induction dose. A “maintenance dose” can be a single dose or, alternatively, a set of doses. A “maintenance dose” is administered during the maintenance period of therapy.

As used herein, the term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a human IL-23. For example, antibody fragments capable of binding to IL-12/23 or portions thereof, including, but not limited to, Fab (e.g. by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g. by molecular biology techniques) fragments, are encompassed by the present invention (see, e.g. Colligan et al., Current Protocols in Immunology, John Wiley & Sons, NY, NY, (1994-2001)).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the CH1 domain and/or hinge region of the heavy chain. The various portions of antibodies can be joined together chemically by conventional techniques, or can be prepared as a contiguous protein using genetic engineering techniques.

As used herein “an antibody that binds to the p19 subunit of human IL-23” refers to an antibody that binds to the p19 subunit of human IL-23 but does not bind to the p40 subunit of human IL-23. An “antibody that binds to the p19 subunit of human IL-23” thus binds to human IL-23 but does not bind to human IL-12.

Mirikizumab, CAS Registry No. 1884201-71-1, is an engineered, IgG₄-kappa monoclonal antibody targeting the p19 subunit of human IL-23. The antibody and methods of making same are described in U.S. Pat. No. 9,023,358.

The antibody that binds to the p19 subunit of human IL-23, or pharmaceutical compositions comprising the same, may be administered by parenteral routes (e.g., subcutaneous, intravenous, intraperitoneal, intramuscular, or transdermal).

The term “intravenous infusion” refers to introduction of an agent into the vein of an animal or human patient over a period of time greater than approximately 15 minutes, generally between approximately 30 to 90 minutes.

The term “subcutaneous injection” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

Pharmaceutical compositions comprising an anti-IL-23p19 antibody for use in the methods of the present invention can be prepared by methods well known in the art (e.g., Remington: The Science and Practice a/Pharmacy, 19^{th} edition (1995), (A. Gennaro et al., Mack Publishing Co.) and comprise an antibody as disclosed herein, and one or more pharmaceutically acceptable carriers, diluents, or excipients.

In one aspect, the present invention provides a method for the treatment of psoriasis comprising administering mirikizumab to a patient, said method comprising:
a) administering at least one induction dose of mirikizumab to the patient, wherein the induction dose comprises 20 mg to 600 mg of mirikizumab; and
b) administering at least one maintenance dose of mirikizumab to the patient after the last induction dose is administered, wherein the maintenance dose comprises 20 mg to 600 mg of mirikizumab.

In a further aspect, the present invention provides provides a method of treating psoriasis comprising administering mirikizumab to a patient, said method comprising:
a) administering one or more inductions dose(s) of mirikizumab to the patient during an induction period, wherein the induction dose comprises 20-600 mg of mirikizumab;
c) determining the disease activity level of the patient at the end of the induction period and
   i) administering one or more maintenance dose(s) to a patient that has not achieved a high level of clinical response at the end of the induction period, wherein the one or more maintenance dose(s) each comprise 20 mg to 600 mg of mirikizumab; and
   ii) continuing assessment of the disease activity level of a patient that has achieved a high level of clinical response beyond the induction period and administering one or more maintenance dose(s) to the patient if the patient's disease activity level falls below a high level of clinical response, wherein the one or more maintenance dose(s) are administered until the patient re-achieves a high level of clinical response, and wherein the one or more maintenance dose(s) each comprise 20 mg to 600 mg of mirikizumab.

In a still further aspect, the present invention provides a method of treating psoriasis comprising administering mirikizumab to a patient, said method comprising:
i) administering one or more induction dose(s) of mirikizumab until the patient achieves clinical remission, wherein the one or more induction dose(s) each comprise 20 mg to 600 mg of mirikizumab; and
ii) monitoring the disease activity level of the patient and administering one or more maintenance dose(s) of mirikizumab if the disease activity of the patient falls below clinical remission, wherein the one or more maintenance dose(s) are administered until the patient re-achieves clinical remission, and wherein the one or more maintenance dose(s) each comprise 20 mg to 600 mg of mirikizumab.

Preferred embodiments of the present invention have been described hereinabove. Representative examples of doses and dose regimens according to the present invention are described in Table 2.

TABLE 2
Doses and dose regimen

| | (i) Induction | | | Alternative |
|---|---|---|---|---|
| | Period and (ii) | | Frequency of | frequencies of |
| Induction | Frequency of | Maintenance | maintenance | maintenance |
| Dose(mg) | Induction Dose | Dose(s) | dose(s) | dose(s) |
| 30 | (i) 12 weeks | 30 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 30 | (i) 12 weeks | 300 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 30 | (i) 16 weeks | 30 | First: 4 weeks after | Further: Q8W, |
| | (ii) Q4W (Weeks | | last induction dose | Q12W |
| | 0, 4, 8 and 12) | | (Week 16); | |
| | | | Further: Q4W | |
| 30 | (i) 16 weeks | 300 | First: 4 weeks after | Further: Q8W, |
| | (ii) Q4W (Weeks | | last induction dose | Q12W |
| | 0, 4, 8 and 12) | | (Week 16); | |
| | | | Further: Q4W | |
| 30 | (i) 16 weeks | 30 | First: 8 weeks after | Further: Q4W, |
| | (ii) Q8W (Weeks | | last induction dose | Q12W |
| | 0 and 8) | | (Week 16); | |
| | | | Further: Q8W | |
| 30 | (i) 16 weeks | 300 | First: 8 weeks after | Further: Q4W, |
| | (ii) Q8W (Weeks | | last induction dose | Q12W |
| | 0, and 8) | | (Week 16); | |
| | | | Further: Q8W | |
| 30 | (i) 12 weeks | 300 | Regimen for | Further: Q8W, |
| | (ii) Q4W (Weeks | | patients that have a | Q12W |
| | 0, 4 and 8) | | disease activity | |
| | | | level of <PASI 90 | |
| | | | or <sPGA (0, 1) at | |
| | | | Week 12 | |
| | | | First: 4 weeks after | |
| | | | last induction dose | |
| | | | (Week 12); | |
| | | | Further: Q4W | |
| 30 | (i) 12 weeks | 30 | Regimen for | Further: Q8W, |
| | (ii) Q4W (Weeks | | patients that have a | Q12W |
| | 0, 4 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q4W until | |
| | | | the disease activity | |
| | | | level of the patient | |
| | | | is ≥PASI 90 | |
| | | | or ≥sPGA (0, 1) | |
| 30 | (i) 16 weeks | 30 | Regimen for | Further: Q8W, |
| | (ii) Q4W (Weeks | | patients that have a | Q12W |
| | 0, 4, 8 and 12) | | disease activity | |
| | | | level of <PASI 90 | |
| | | | or <sPGA (0, 1) at | |
| | | | Week 12 | |
| | | | First: 4 weeks after | |
| | | | last induction dose | |
| | | | (Week 16); | |
| | | | Further: Q4W | |
| 30 | (i) 16 weeks | 300 | Regimen for | Further: Q8W, |
| | (ii) Q4W (Weeks | | patients that have a | Q12W |
| | 0, 4, 8 and 12) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (2-5); | |
| | | | Further: Q4W until | |
| | | | the disease activity | |
| | | | level of the patient | |
| | | | is ≥PASI 90 | |
| | | | or ≥sPGA (0, 1) | |
| | | | First: 4 weeks after | |
| | | | last induction dose; | |
| | | | Further: Q4W | |
| 30 | (i) 16 weeks | 300 | Regimen for | Further: Q4W, |
| | (ii) Q8W (Weeks | | patients that have a | Q12W |
| | 0 and 8) | | disease activity | |
| | | | level of <PASI 90 | |
| | | | or <sPGA (0, 1) at | |
| | | | Week 16 | |
| | | | First: 8 weeks after | |
| | | | last induction dose | |
| | | | (Week 16); | |
| | | | Further: Q8W | |
| 30 | (i) 16 weeks | 30 | Regimen for | Further: Q4W, |
| | (ii) Q8W (Weeks | | patients that have a | Q12W |
| | 0 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q8W until | |
| | | | the disease activity | |
| | | | level of the patient | |
| | | | is ≥PASI 90 | |
| | | | or ≥sPGA (0, 1) | |
| 100 | (i) 12 weeks | 100 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 100 | (i) 12 weeks | 300 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 100 | (i) 16 weeks | 100 | First: 4 weeks after | Further: Q8W, |
| | (ii) Q4W (Weeks | | last induction dose | Q12W |
| | 0, 4, 8 and 12) | | (Week 16); | |
| | | | Further: Q4W | |
| 100 | (i) 16 weeks | 300 | First: 4 weeks after | Further: Q8W, |
| | (ii) Q4W (Weeks | | last induction dose | Q12W |
| | 0, 4 and 8) | | (Week 16); | |
| | | | Further: Q4W | |
| 100 | (i) 16 weeks | 100 | First: 8 weeks after | Further: Q4W, |
| | (ii) Q8W (Weeks | | last induction dose | Q12W |
| | 0 and 8) | | (Week 16); | |
| | | | Further: Q8W | |
| 100 | (i) 16 weeks | 300 | First: 8 weeks after | Further: Q4W, |
| | (ii) Q8W (Weeks | | last induction dose | Q12W |
| | 0 and 8) | | (Week 16); | |
| | | | Further: Q8W | |
| 100 | (i) 12 weeks | 300 | Regimen for | Further: Q8W, |
| | (ii) Q4W (Weeks | | patients that have a | Q12W |
| | 0, 4 and 8) | | disease activity | |
| | | | level of <PASI 90 | |
| | | | or <sPGA (0, 1) at | |
| | | | Week 12 | |
| | | | First: 4 weeks after | |
| | | | last induction dose | |
| | | | (Week 12); | |
| | | | Further: Q4W | |
| 100 | (i) 12 weeks | 100 | Regimen for | Further: Q8W, |
| | (ii) Q4W (Weeks | | patients that have a | Q12W |
| | 0, 4 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q4W until | |
| | | | the disease activity | |
| | | | level of the patient | |
| | | | is ≥PASI 90 | |
| | | | or ≥sPGA (0, 1) | |
| 100 | (i) 16 weeks | 300 | Regimen for | Further: Q8W, |
| | (ii) Q4W (Weeks | | patients that have a | Q12W |
| | 0, 4, 8 and 12) | | disease activity | |
| | | | level of <PASI 90 | |
| | | | or <sPGA (0, 1) at | |
| | | | Week 16 | |
| | | | First: 4 weeks after | |
| | | | last induction dose | |
| | | | (Week 16); | |
| | | | Further: Q4W | |
| 100 | (i) 16 weeks | 100 | Regimen for | Further: Q8W, |
| | (ii) Q4W (Weeks | | patients that have a | Q12W |
| | 0, 4, 8 and 12) | | disease a patient | |
| | | | that has a disease | |
| | | | activity level | |
| | | | of ≥PASI 90 | |
| | | | or ≥sPGA (0, 1) at | |
| | | | Week 16 | |
| | | | First: PRN - when | |
| | | | the patient's disease | |
| | | | activity level | |
| | | | is <PASI 90 | |
| | | | or <sPGA (0, 1); | |
| | | | Further: Q4W until | |
| | | | the disease activity | |
| | | | level of the patient | |
| | | | is ≥PASI 90 | |
| | | | or ≥sPGA (0, 1) | |
| | | | First: 4 weeks after | |
| | | | last induction dose; | |
| | | | Further: Q4W | |
| 100 | (i) 16 weeks | 300 | Regimen for | Further: Q4W, |
| | (ii) Q8W (Weeks | | patients that have a | Q12W |
| | 0 and 8) | | disease activity | |
| | | | level of <PASI 90 | |
| | | | or <sPGA (0, 1) at | |
| | | | Week 16 | |
| | | | First: 4 weeks after | |
| | | | last induction dose | |
| | | | (Week 16); | |
| | | | Further: Q8W | |
| 100 | (i) 16 weeks | 100 | Regimen for | Further: Q4W, |
| | (ii) Q8W (Weeks | | patients that have a | Q12W |
| | 0 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 or sPGA | |
| | | | (0, 1); | |
| | | | Further: Q8W until | |
| | | | the disease activity | |
| | | | level of the patient | |
| | | | is ≥PASI 90 | |
| | | | or ≥sPGA (0, 1) | |
| 250 | (i) 12 weeks | 125 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 250 | (i) 12 weeks | 250 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 250 | (i) 16 weeks | 125 | First: 4 weeks after | Further: Q8W, |
| | (ii) Q4W (Weeks | | last induction dose | Q12W |
| | 0, 4, 8 and 12) | | (Week 16); | |
| | | | Further: Q4W | |
| 250 | (i) 16 weeks | 250 | First: 4 weeks after | Further: Q8W, |
| | (ii) Q4W (Weeks | | last induction dose | Q12W |
| | 0, 4 and 8) | | (Week 16); | |
| | | | Further: Q4W | |
| 250 | (i) 16 weeks | 125 | First: 8 weeks after | Further: Q4W, |
| | (ii) Q8W (Weeks | | last induction dose | Q12W |
| | 0 and 8) | | (Week 16); | |
| | | | Further: Q8W | |
| 250 | (i) 16 weeks | 250 | First: 8 weeks after | Further: Q4W, |
| | (ii) Q8W (Weeks | | last induction dose | Q12W |
| | 0, and 8) | | (Week 16); | |
| | | | Further: Q8W | |
| 250 | (i) 12 weeks | 250 | Regimen for | Further: Q4W, |
| | (ii) Q4W (Weeks | | patients that have a | Q12W |
| | 0, 4 and 8) | | disease activity | |
| | | | level of <PASI 90 | |
| | | | or <sPGA (0, 1) at | |
| | | | Week 12 | |
| | | | First: 4 weeks after | |
| | | | last induction dose | |
| | | | (Week 12); | |
| | | | Further: Q8W | |
| 250 | (i) 12 weeks | 125 or 250 | Regimen for | Further: Q4W, |
| | (ii) Q4W (Weeks | | patients that have a | Q12W |
| | 0, 4 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q8W until | |
| | | | the disease activity | |
| | | | level of the patient | |
| | | | is ≥PASI 90 | |
| | | | or ≥sPGA (0, 1) | |
| 250 | (i) 16 weeks | 250 | Regimen for | Further: Q4W, |
| | (ii) Q4W (Weeks | | patients that have a | Q12W |
| | 0, 4, 8 and 12) | | disease activity | |
| | | | level of <PASI 90 | |
| | | | or <sPGA (0, 1) at | |
| | | | Week 16 | |
| | | | First: 4 weeks after | |
| | | | last induction dose | |
| | | | (Week 16); | |
| | | | Further: Q8W | |
| 250 | (i) 16 weeks | 125 or 250 | Regimen for | Further: Q4W, |
| | (ii) Q4W (Weeks | | patients that have a | Q12W |
| | 0, 4, 8 and 12) | | disease a patient | |
| | | | that has a disease | |
| | | | activity level | |
| | | | of ≥PASI 90 | |
| | | | or ≥sPGA (0, 1) at | |
| | | | Week 16 | |
| | | | First: PRN - when | |
| | | | the patient's disease | |
| | | | activity level | |
| | | | is <PASI 90 | |
| | | | or <sPGA (2-5); | |
| | | | Further: Q4W until | |
| | | | the disease activity | |
| | | | level of the patient | |
| | | | is ≥PASI 90 | |
| | | | or ≥sPGA (0, 1) | |
| | | | First: 4 weeks after | |
| | | | last induction dose; | |
| | | | Further: Q8W | |
| 250 | (i) 16 weeks | 250 | Regimen for | Further: Q4W, |
| | (ii) Q8W (Weeks | | patients that have a | Q12W |
| | 0 and 8) | | disease activity | |
| | | | level of <PASI 90 | |
| | | | or <sPGA (0, 1) at | |
| | | | Week 16 | |
| | | | First: 8 weeks after | |
| | | | last induction dose | |
| | | | (Week 16); | |
| | | | Further: Q8W | |
| 250 | (i) 16 weeks | 125 or 250 | Regimen for | Further: Q4W, |
| | (ii) Q8W (Weeks | | patients that have a | Q12W |
| | 0 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q8W until | |
| | | | the disease activity | |
| | | | level of the patient | |
| | | | is ≥PASI 90 | |
| | | | or ≥sPGA (0, 1) | |
| 300 | (i) 12 weeks | 300 | First: 4 weeks after | Further: Q8W, |
| | (ii) Q4W (Weeks | | last induction dose | Q12W |
| | 0, 4 and 8) | | (Week 12); | |
| | | | Further: Q4W | |
| 300 | (i) 16 weeks | 300 | First: 4 weeks after | Further: Q8W, |
| | (ii) Q4W (Weeks | | last induction dose | Q12W |
| | 0, 4, 8 and 12) | | (Week 12); | |
| | | | Further: Q4W | |
| 300 | (i) 16 weeks | 300 | First: 8 weeks after | Further: Q4W, |
| | (ii) Q8W (Weeks | | last induction dose | Q12W |
| | 0 and 8) | | (Week 16); | |
| | | | Further: Q8W | |
| 300 | (i) 12 weeks | 300 | Regimen for | Further: Q8W, |
| | (ii) Q4W (Weeks | | patients that have a | Q12W |
| | 0, 4 and 8) | | disease activity | |
| | | | level of <PASI 90 | |
| | | | or <sPGA (0, 1) at | |
| | | | Week 12 | |
| | | | First: 4 weeks after | |
| | | | last induction dose | |
| | | | (Week 12); | |
| | | | Further: Q4W | |
| 300 | (i) 12 weeks | 300 | Regimen for | Further: Q8W, |
| | (ii) Q4W (Weeks | | patients that have a | Q12W |
| | 0, 4 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 | |
| | | | or <sPGA (0, 1); | |
| | | | Further: Q4W until | |
| | | | the disease activity | |
| | | | level of the patient | |
| | | | is ≥PASI 90 | |
| | | | or ≥sPGA (0, 1) | |
| 300 | (i) 16 weeks | 300 | Regimen for | Further: Q8W, |
| | (ii) Q4W (Weeks | | patients that have a | Q12W |
| | 0, 4, 8 and 12) | | disease activity | |
| | | | level of <PASI 90 | |
| | | | or <sPGA (0, 1) at | |
| | | | Week 16 | |
| | | | First: 4 weeks after | |
| | | | last induction dose; | |
| | | | Further: Q4W | |
| 300 | (i) 16 weeks | 300 | Regimen for | Further: Q8W, |
| | (ii) Q4W (Weeks | | patients that have a | Q12W |
| | 0, 4, 8 and 12) | | disease a patient | |
| | | | that has a disease | |
| | | | activity level | |
| | | | of ≥PASI 90 | |
| | | | or ≥sPGA (0, 1) at | |
| | | | Week 16 | |
| | | | First: PRN - when | |
| | | | the patient's disease | |
| | | | activity level | |
| | | | is <PASI 90 | |
| | | | or <sPGA (0, 1); | |
| | | | Further: Q4W until | |
| | | | the disease activity | |
| | | | level of the patient | |
| | | | is ≥PASI 90 | |
| | | | or ≥sPGA (2-5) | |
| 300 | (i) 16 weeks | 300 | Regimen for | Further: Q4W, |
| | (ii) Q8W (Weeks | | patients that have a | Q12W |
| | 0 and 8) | | disease activity | |
| | | | level of <PASI 90 | |
| | | | or <sPGA (0, 1) at | |
| | | | Week 16 | |
| | | | First: 8 weeks after | |
| | | | last induction dose | |
| | | | (Week 16); | |
| | | | Further: Q8W | |
| 300 | (i) 16 weeks | 300 | Regimen for | Further: Q4W, |
| | (ii) Q8W (Weeks | | patients that have a | Q12W |
| | 0 and 8) | | disease a patient | |
| | | | that has a disease | |
| | | | activity level | |
| | | | of ≥PASI 90 | |
| | | | or ≥sPGA (0, 1) at | |
| | | | Week 12 | |
| | | | First: PRN - when | |
| | | | the patient's disease | |
| | | | activity level | |
| | | | is <PASI 90 or sPGA | |
| | | | (0, 1); | |
| | | | Further: Q8W until | |
| | | | the disease activity | |
| | | | level of the patient | |
| | | | is ≥PASI 90 | |
| | | | or ≥sPGA (0, 1) | |

### EXAMPLES

### Example 1: Clinical Study

### Overview

This study is a Phase II study multicenter, randomized, parallel-arm, placebo-controlled trial in subjects with moderate or severe plaque psoriasis. The study is designed to determine whether subcutaneous (SC) administration of mirikizumab, is safe and efficacious in subjects with moderate to severe plaque psoriasis. The study comprises a screening period of up to a maximum of 28 days, a 16-week double-blinded SC therapy period, an 88-week SC therapy for responders and non-responders at Week 16, and a 16-week follow-up period.

### Objectives

The primary objective of the study is to test the hypothesis that treatment with mirikizumab is superior to placebo in inducing PASI 90 response at Week 16 in subjects with moderate to severe plaque psoriasis. Secondary objectives included the following:
To evaluate the safety and tolerability of treatment with mirikizumab;
To evaluate the efficacy of treatment with mirikizumab compared to placebo in inducing PASI 100 and PASI 75 at Week 16
To evaluate the efficacy of treatment with mirikizumab compared to placebo in inducing sPGA 0 (clear) and sPGA 0/1 at week 16;
To characterize the long term efficacy of mirikizumab on the PASI 100, PASI 90 and PASI 75 responses at Week 52, 104 and 120; and
To characterize the PK of mirikizumab
   The endpoints of the study include the following:
The proportion of subjects achieving PASI 90 at Week 16;
Adverse event and discontinuation rates;
The proportion of subjects achieving PASI 100 and PASI 75 at Week 16;
The proportion of subjects achieving sPGA 0 and sPGA 0/1 at Week 16;
The proportion of subjects achieving PASI 100, PASI 90 and PASI 75 at Weeks 52, 104 and 120; and
Clearance and volume of distribution.

Adverse events were coded according to the Medical Dictionary for Regulatory Activities (MedDRA) Version 19.1 and summarized by system organ class, preferred term, severity and relationship to investigational product. A treatment-emergent AE (TEAE) was defined as an event that first occurred or worsened in severity after baseline. The Columbia-Suicide Severity Rating Scale (C-SSRS; Columbia University Medical Center [WWW]) was used to capture the occurrence, severity and frequency of suicide-related ideations and behaviours.

### Methods

The study comprises a screening period, two treatment periods for patients that achieve PASI 90 at Week 16 (a 16-week double-blinded SC induction therapy period and an 88-week SC maintenance therapy period) and two treatment periods for patients that do not achieve PASI 90 at Week 16 (a 16-week double-blinded SC induction therapy period and an 88-week SC maintenance therapy period). The maintenance period is followed by a 16-week follow-up period to assess subject safety and study drug efficacy.

Approximately 40% of the patients randomized to study treatment have previously been exposed to at least one biologic therapy (anti-TNF biologic, or anti-IL-17 targeting biologics) and approximately 60% of the patients are naive to biologic therapy.

### a) Screening Period

Subjects are evaluated for study eligibility ≤28 days before the baseline visit. At the baseline visit, subjects who fulfill the eligibility criteria will be randomized to 1 of 4 induction treatment arms.

Inclusion criteria for this study included adult patients (18-75 years of age), with an investigator-confirmed diagnosis of chronic plaque psoriasis vulgaris for at least 6 months prior to baseline. Patients must have had ≥10% body surface area (BSA) involvement, absolute PASI score ≥12 and static Physician's Global Assessment (sPGA) score of ≥3 at screening and baseline, and they must have been deemed eligible for biologic therapy for psoriasis. Anti-tumour necrosis factor (anti-TNF) or anti-IL-17 biologic use within 8 weeks of baseline was not allowed. Previous exposure to any biologic therapy targeting IL-23 was also not allowed, with the exception of briakinumab. Patients were to maintain stable dosages of their usual medication regimens for concomitant conditions or diseases throughout the study unless those medications were specifically excluded in the protocol, or if a change was required to treat an adverse event (AE). Topical steroids were permitted for use limited to the face, axilla and/or genitalia, as needed, except for 24 hours prior to study visits.

### b) Induction Period

A double-blind 16-week induction period is designed to establish the efficacy and safety of mirikizumab administered at Week 0 and Week 8. At Week 0 (baseline), patients are enrolled into to one of four induction treatment arms (placebo, 30 mg mirikizumab SC, 100 mg mirikizumab SC, and 300 mg mirikizumab SC) to adequately evaluate the study endpoints. Patients enrolled in the trial are stratified across the treatment arms on the basis of previous exposure to biologic therapy for treatment of psoriasis. Blinded study drug (mirikizumab or placebo) is administered at Weeks 0 and 8.

### c) Maintenance Period

The maintenance period consists of 88 weeks of treatment. At the end of the induction period (Week 16) subjects continue treatment in the maintenance period in one of two treatment arms through Week 104. All placebo subjects and subjects assigned to treatment with mirikizumab that have a <PASI 90 at Week 16 receive mirikizumab 300 mg SC Q8W during the entire maintenance period. Subjects with ≥PASI 90 at Week 16 (PRN dosing group) are dosed with mirikizumab at the baseline dose level assignment no more frequently than Q8W when disease activity level is <PASI 90, and this treatment continues until ≥PASI 90 is regained.

Subjects in the maintenance PRN dosing arm may receive blinded rescue treatment with 300 mg Q8W if not regaining a PASI ≥90 after 3 consecutive doses of retreatment, or any subject who is below PASI50 following one retreatment dose.

### d) Follow-Up Period

The follow-up period will include a visit every 4 weeks for a total of 16 weeks following Week 104 to assess subject safety and study drug efficacy.

### Statistical Analyses

Assuming 60% and 3% PASI 90 response rates at 16 weeks for mirikizumab and placebo, respectively, pairwise comparisons to placebo were determined to have over 99% power using a 2-sided Fisher's exact test at the 0.05 significance level, with no adjustment for multiple comparisons. All randomized patients were analysed according to the dose group to which they were assigned (intent to treat). Safety analyses were performed for all patients who received at least one dose of study drug.

A logistic regression analysis with treatment, geographic region (United States/Outside United States [US/OUS]) and previous biologic therapy (yes/no), was used for the comparison of each mirikizumab dose regimen (300 mg, 100 mg 30 mg) and placebo for categorical binary efficacy and health outcome variables.

### Results: Study Population

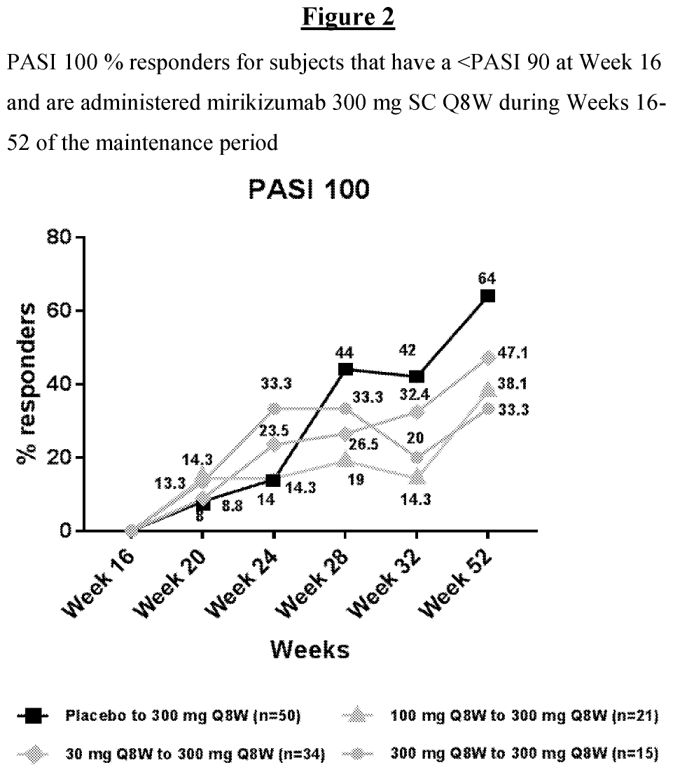

Amongst 251 patients screened, 205 were randomized to receive placebo (N=52), mirikizumab Q8W 30 mg (N=51), mirikizumab Q8W 100 mg (N=51) and mirikizumab Q8W 300 mg (N=51). Ninety-seven percent of patients completed the initial 16-week period of this study (FIG. 2). Patients generally had similar baseline characteristics across treatment groups. On average, patients were 47 years of age, body weight 89 kg and had been diagnosed with psoriasis for 19 years. There were more male patients in all treatment groups and approximately 41% of patients had previously been treated with biologic therapy. On average, patients had a baseline PASI score of 20 with 25% BSA affected by psoriasis.

### Results: Efficacy

At Week 16, a statistically significantly higher proportion of patients achieved a PASI 90 response (primary outcome) in the 30 mg (29.4%, p=0.009), 100 mg (58.8%, p<0.001) and 300 mg (66.7%, p<0.001) mirikizumab groups compared to placebo (0%) (Table 3).

Additionally, Week 16 PASI 75 and sPGA 0/1 response rates were, respectively, 52.9% and 37.3% in the 30 mg, 78.4% and 70.6% in the 100 mg, and 74.5% and 68.6% in the 300 mg mirikizumab dose groups compared to 3.8% and 1.9% in the placebo group (p<0.001 for each mirikizumab dose group vs. placebo). PASI 100 and sPGA 0 response rates were identical at Week 16, with 15.7% in the 30 mg, 31.4% in the 100 mg and 31.4% in the 300 mg mirikizumab groups compared to 0% in the placebo group (p=0.039 for 30 mg vs. placebo; p=0.007 for the higher dose groups vs. placebo) achieving complete clearance of psoriasis (Table 3). The proportion of patients with complete clearance of scalp psoriasis, as measured by PSSI=0, was 43.1% in the 30 mg, 74.5% in the in the 100 mg and 51.0% in the 300 mg mirikizumab groups compared to 5.8% in the placebo group (p<0.001 for each mirikizumab dose group vs placebo) (Table 2). For all Week 16 outcomes presented here, the highest responses were seen in the mirikizumab 100 mg and 300 mg treatment groups.

Similarly high response rates were observed for absolute PASI thresholds. At least 80% of patients treated with mirikizumab 100 mg or 300 mg had PASI scores of 5 or less and at least 70% had PASI scores of 3 or less at Week 16. More than 50% of the patients treated with mirikizumab 300 mg had an absolute PASI score of 1 or less. In addition, more than 50% of the patients treated with mirikizumab 100 mg or 300 mg had no more than 1% of their BSA covered with psoriasis at Week 16 (Table 3).

At Week 16, the proportion of patients reporting no symptoms of itching, pain, burning or stinging (PSS Symptoms domain score=0) and no impact of their psoriasis on their quality of life (DLQI 0/1) was significantly higher for each mirikizumab treatment groups versus placebo, with the highest response rates noted in the 100 mg and 300 mg treatment groups (Table 3).

In the biologic-naive and prior biologic therapy populations, PASI 90 response rates improved in 100 mg (66.7%, 47.6%; p=0.001), 300 mg (69.0%, 63.6%; p=0.001), and 30 mg (38.7%, 15.0%; p=0.013) vs. placebo (0%). Similarly, PASI 100 response rates in both the populations improved in 100 mg (36.7%, 23.8%; p=0.016), 300 mg (31.0%, 31.8%; p=0.025), and 30 mg (22.6%, 5.0%, p=0.050) vs. placebo (0%). Also, PASI 75 response rates were significantly higher with 100 mg Q8W and 300 mg Q8W as compared with placebo for Naive (80.0% vs. 6.5% and 72.4% vs. 6.5%; p<0.001) and Prior (76.2% vs. 0% and 77.3% vs. 0%; p<0.001) patient populations. Similar results were found with 30 mg Q8W vs. placebo for both the patient populations (Naive: 61.3% vs. 6.5%; Prior: 40.0% vs. 0%; p<0.001). In naive patient population, sPGA (0,1) response rate significantly improved in 100 mg Q8W (80.6%), 300 mg Q8W (69.0%), and 30 mg Q8W (48.4%) vs. placebo (3.2%) (p<0.001). sPGA (0,1) response rate in prior biologic therapy patient population was also significantly higher in 100 mg Q8W (55.0%; p<0.001), 300 mg Q8W (68.2%; p<0.001), and 30 mg Q8W (20.0%; p<0.05) vs. placebo (0%).

TABLE 3
Study outcomes at Week 16

| NRI and n (%) | | | Mirikizumab | | Mirikizumab | | Mirikizumab | |
|---|---|---|---|---|---|---|---|---|
| unless | | | Q8W | | Q8W | | Q8W | |
| otherwise | Placebo | | 30 mg | | 100 mg | | 300 mg | |
| specified | (N = 52) | | (N = 51) | | (N = 51) | | (N = 51) | |
| PASI score | 19.5 | (8.4) | 6.0 | (5.6)*** | 2.7 | (4.2)*** | 2.5 | (4.2)*** |
| (observed), | | | | | | | | |
| mean (SD) | | | | | | | | |
| PASI 100 | 0 | | 8 | (15.7)* | 16 | (31.4)** | 16 | (31.4)** |
| PASI 90 | 0 | | 15 | (29.4)** | 30 | (58.8)*** | 34 | (66.7)*** |
| PASI 75 | 2 | (3.8) | 27 | (52.9)*** | 40 | (78.4)*** | 38 | (74.5)*** |
| PASI ≤1 | 0 | | 8 | (15.7)* | 23 | (45.1)** | 27 | (52.9)*** |
| PASI ≤3 | 2 | (3.8) | 21 | (41.2)*** | 37 | (72.5)*** | 36 | (70.6)*** |
| PASI ≤5 | 2 | (3.8) | 28 | (54.9)*** | 41 | (80.4)*** | 41 | (80.4)*** |
| sPGA 0/1 | 1 | (1.9) | 19 | (37.3)*** | 36 | (70.6)*** | 35 | (68.6)*** |
| sPGA 0 | 0 | | 8 | (15.7)* | 16 | (31.4)** | 16 | (31.4)** |
| BSA 0/1 | 1 | (1.9) | 10 | (19.6)* | 28 | (54.9)*** | 30 | (58.8)*** |
| PSSI = 0 | 3 | (5.8) | 22 | (43.1)*** | 38 | (74.5)*** | 26 | (51.0)*** |
| PSS Symptoms | 0 | | 8 | (15.7)* | 14 | (27.5)* | 16 | (31.4)** |
| domain | | | | | | | | |
| score = 0 | | | | | | | | |
| DLQI 0/1 | 2 | (3.8) | 18 | (35.3)*** | 25 | (49.0)*** | 24 | (47.1)*** |

*p < 0.05;
**p < 0.01;
***p < 0.001 vs. placebo.
BMI = Body Mass Index; NRI = non-responder imputation; PASI = Psoriasis Area and Severity Index; PASI 75 = 75% reduction in the Psoriasis Area and Severity Index; PASI 90 = 90% improvement in Psoriasis Area and Severity Index; PASI 100 = 100% improvement in Psoriasis Area and Severity Index; sPGA = static Physician's Global Assessment; BSA = Body Surface Area; PSSI = Psoriasis Scalp Severity Index; PSS = Psoriasis Symptom Scale; DLQI = Dermatology Life Quality Index.

All placebo subjects and subjects assigned to treatment with mirikizumab that have a <PASI 90 at Week 16 receive mirikizumab 300 mg SC Q8W during the entire maintenance period.

At Week 52, after 36 weeks on mirikizumab 300 mg maintenance dose, 82.0% (n=41) and 64.0% (n=32) of patients in the placebo/300 mg group achieved PASI 90 and 100, respectively.

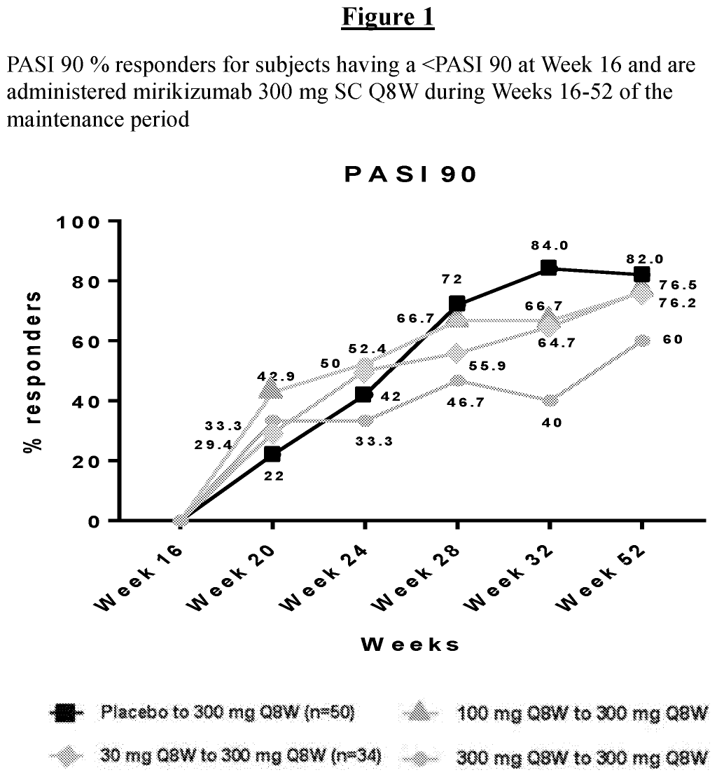

Among those who did not achieve PASI 90 at Week 16 and entered the maintenance period of the study, 76.5% (n=26), 76.2% (n=16), and 60.0% (n=9) of patients in the mirikizumab 30 mg/300 mg (N=34), 100 mg/300 mg (N=21), and 300 mg/300 mg (N=15) groups, respectively, achieved PASI 90 at Week 52 (FIG. 1).

PASI 100 was achieved by 47.1% (n=16), 38.1% (n=8), and 33.3% (n=5) patients in the mirikizumab 30 mg/300 mg, 100 mg/300 mg, and 300 mg/300 mg groups, respectively, at Week 52 (FIG. 2).

Subjects with ≥PASI 90 at Week 16 (PRN dosing group) are dosed with mirikizumab at the baseline dose level assignment no more frequently than Q8W when disease activity level is <PASI 90, and this treatment continues until ≥PASI 90 is regained.

After Week 16, median time to loss of PASI 90 response was 15.7 weeks with mirikizumab 30 mg, 11.8 weeks with mirikizumab 100 mg, and 16.3 weeks with mirikizumab 300 mg. Median time to loss of PASI 100 response was 14.1 weeks with mirikizumab 30 mg, 8.1 weeks with mirikizumab 100 mg, and 12.1 weeks with mirikizumab 300 mg. Four weeks after retreatment, 78.6% of mirikizumab 30 mg, 65.4% of mirikizumab 100 mg, and 80.0% of mirikizumab 300 mg patients recaptured PASI 90 and 0% of mirikizumab 30 mg, 12.5% of mirikizumab 100 mg, and 35.7% of mirikizumab 300 mg patients recaptured PASI 100. Eight weeks after retreatment, 92.9% of mirikizumab 30 mg, 88.5% of mirikizumab 100 mg, and 96.7% of mirikizumab 300 mg patients recaptured PASI 90.

Mirikizumab, given every 8 weeks as 100 mg or 300 mg subcutaneous injections resulted in the majority of patients achieving clear or nearly clear skin after 16 weeks of induction treatment and again after 32 weeks of maintenance therapy. Response rates for all efficacy outcomes were statistically significantly higher for all mirikizumab treatment arms vs. placebo, and highest in the mirikizumab 100 mg and mirikizumab 300 mg treatment arms. Even though almost all patients in this trial had scalp psoriasis at baseline, more than 50% of patients in the mirikizumab 300 mg group and nearly 75% of patients in the mirikizumab 100 mg group treatment arm had no evident scalp psoriasis at Week 16.

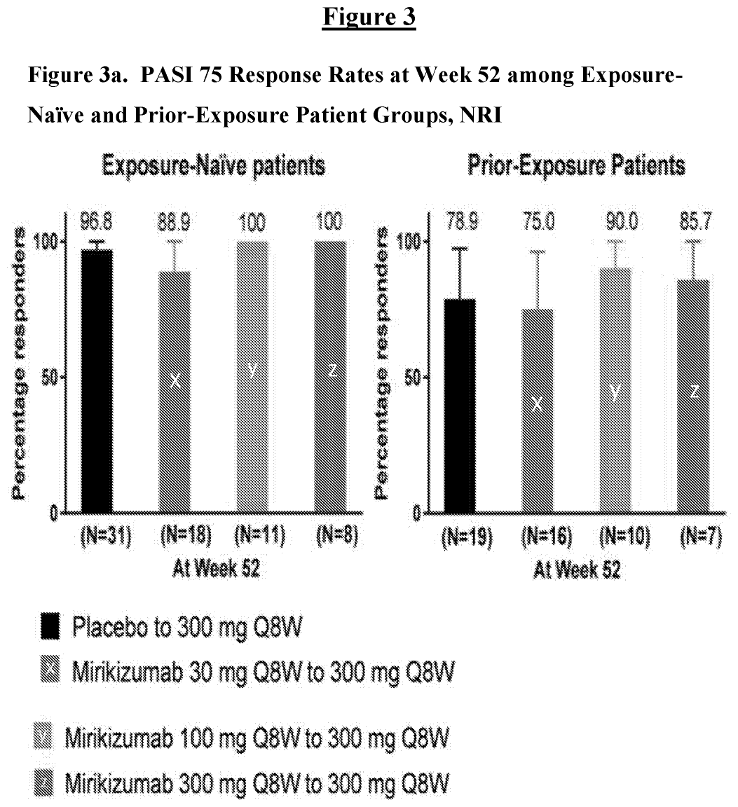

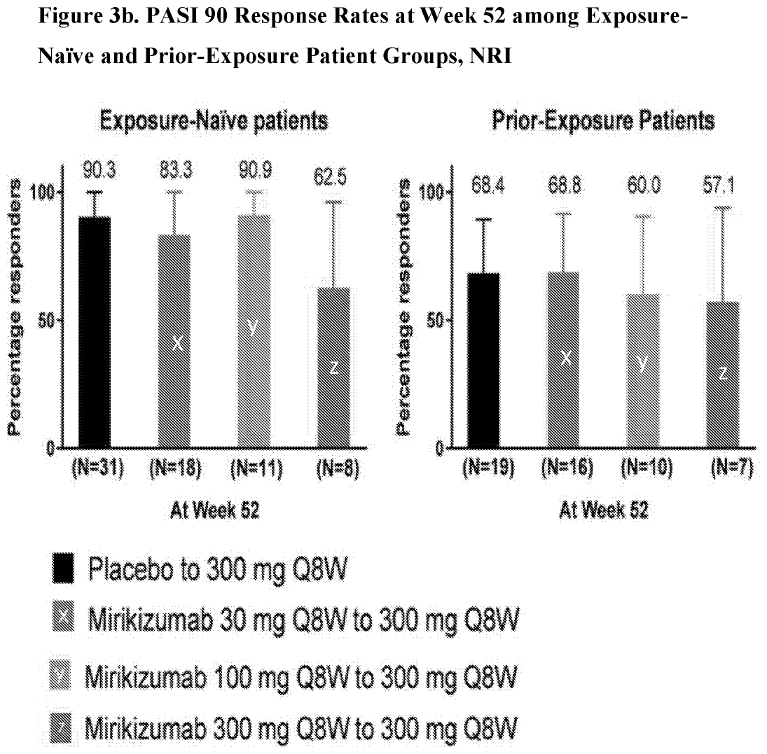

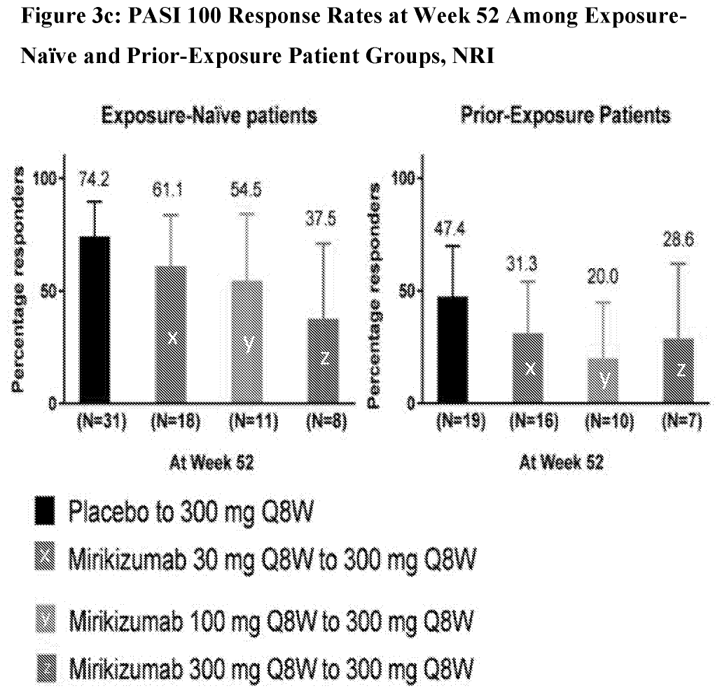

Long-term treatment (Weeks 16-52) with mirikizumab substantially improved disease activity in both exposure-naïve and prior-exposure-to-biologics patients with moderate-to-severe plaque psoriasis who did not achieve PASI 90 at Week 16 (See FIGS. 3a, 3b and 3c. The results demonstrate that mirikizumab is effective in achieving high PASI 90 response among patients who received prior biologic therapy

### Results: Safety

Percentages of patients reporting at least one TEAE were comparable across treatment arms during the first 16 weeks of this study. The specific event of hypertension was reported in 100 mg (3 patients) and 300 mg (2 patients) dose groups, but not placebo or 30 mg groups. All of these patients had elevated or borderline elevated blood pressure at screening or baseline; two had pre-existing hypertension for which they were being treated. None of these events were serious and none led to discontinuation. Patient incidence rate of infections were also comparable across all treatment arms (Table 4). The most common AEs (at least 3 patients [≥>5%] in any treatment group) included viral upper and other respiratory tract infections, injection-site pain, hypertension and diarrhea.

During the first 16 weeks of the trial, no deaths were reported and there were no major adverse cardiac events or malignancies. Three patients reported serious AEs (SAEs) during the initial 16-week period of the trial. One patient in a mirikizumab group and 1 patient in the placebo group had an SAE of suicidal ideation. In both cases, each patient had a history of psychiatric conditions. Despite improvement with psychiatric treatment, both patients discontinued the study. The third patient who reported an SAE was hospitalised due to increased alanine aminotransferase and aspartate aminotransferase at a study visit. Both test results were >10×ULN (upper limit of normal). The patient had a history of hypercholesterolemia and alcohol abuse several years before starting the study. Other clinical chemistries were within normal limits (bilirubin and alkaline phosphatase) and serology was negative for active or acute hepatitis A, B, or C infection. The patient was completely asymptomatic and denied alcohol use. The patient's liver enzymes returned to normal following therapy with oral phospholipids; however, the investigator decided to discontinue the patient from the study. Following discontinuation, the patient was reported to have resumed alcohol abuse, and liver enzymes were again elevated at a follow up visit.

TABLE 4
Adverse Events

| | | | Mirikizumab | | Mirikizumab | | Mirikizumab | | | |
|---|---|---|---|---|---|---|---|---|---|---|
| | | | Q8W | | Q8W | | Q8W | | Mirikizumab | |
| | Placebo | | 30 mg | | 100 mg | | 300 mg | | total | |
| n (%) | (N = 52) | | (N = 51) | | (N = 51) | | (N = 51) | | (N = 153) | |
| Patients | 25 | (48.1) | 26 | (51.0) | 24 | (47.1) | 24 | (47.1) | 74 | (48.4) |
| with ≥1 | | | | | | | | | | |
| TEAE | | | | | | | | | | |
| Mild | 9 | (17.3) | 18 | (35.3) | 9 | (17.6) | 11 | (21.6) | 38 | (24.8) |
| Moderate | 15 | (28.8) | 7 | (13.7) | 14 | (27.5) | 11 | (21.6) | 32 | (20.9) |
| Severe | 1 | (1.9) | 1 | (2.0) | 1 | (2.0) | 2 | (3.9) | 4 | (2.6) |
| Death | 0 | | 0 | | 0 | | 0 | | 0 | |
| SAEs | 1 | (1.9) | 1 | (2.0) | 0 | | 1 | (2.0) | 2 | (1.3) |
| Investigator- | 7 | (13.5) | 12 | (23.5) | 7 | (13.7) | 9 | (17.6) | 28 | (18.3) |
| defined | | | | | | | | | | |
| treatment- | | | | | | | | | | |
| related AEs | | | | | | | | | | |
| Infections | 12 | (23.1) | 14 | (27.5) | 13 | (25.5) | 13 | (25.5) | 40 | (26.1) |
| Common TEAEs* | | | | | | | | | | |
| Viral URTI | 5 | (9.6) | 5 | (9.8) | 7 | (13.7) | 7 | (13.7) | 19 | (12.4) |
| Other URTI | 2 | (3.8) | 6 | (11.8) | 3 | (5.9) | 2 | (3.9) | 11 | (7.2) |
| Injection- | 1 | (1.9) | 3 | (5.9) | 2 | (3.9) | 2 | (3.9) | 7 | (4.6) |
| site | | | | | | | | | | |
| pain | | | | | | | | | | |

| Hypertension | 0 | | 0 | 3 | (5.9) | 2 | (3.9) | 5 | (3.3) |
|---|---|---|---|---|---|---|---|---|---|
| Diarrhoea | 1 | (1.9) | 0 | 1 | (2.0) | 3 | (5.9) | 4 | (2.6) |

*Common is defined as at least 3 (≥5%) in any treatment group.
TEAE = Treatment-Emergent Adverse Event; SAE = Serious Adverse Event; URTI = Upper Respiratory Tract Infection.

During the maintenance period (Weeks 16-52) of this study, among patients who did not achieve PASI 90 at Week 16, the most common (≥5%) treatment-emergent adverse events (AEs) included nasopharyngitis (n=25; 20.8%), upper respiratory tract infection (n=12; 10.0%), urinary tract infection (n=6; 5.0%), arthralgia (n=8; 6.7%), back pain (n=6; 5.0%), headache (n=6; 5.0%), injection-site pain (n=7; 5.8%), and hypertension. Four (3.3%) patients experienced SAEs and 6 (5.0%) patients discontinued the study due to AEs in this group of patients during Weeks 16-52.

During the maintenance period (Week 16 to 52) of this study, among patients who did achieve PASI 90 at Week 16, treatment-emergent adverse events (AEs) were reported in 67% of the mirikizumab 30 mg cohort, 53% of the mirikizumab 100 mg cohort, and 62% of the mirikizumab 300 mg patients. Two patients reported a serious AE and three patients discontinued due to an AE (n=1, 30 mg mirikizumab; n=2, 100 mg mirikizumab). Across all mirikizumab groups, the most common AEs were nasopharyngitis (10.1%), upper respiratory tract infection (5.1%), and hypertension (5.1%).

### Results: Pharmacokinetics (PK) and Exposure/Response Modelling

### Summary of Exposure/Response Model-Based Analyses

Doses of 30, 100, and 300 mg, administered Q8W SC, provided significant efficacy relative to placebo, with 100 and 300 mg achieving greater efficacy than 30 mg at Week 16. The 300 mg dose provided the highest efficacy for the primary endpoint at Week 16 (PASI 90) and demonstrated a trend towards providing higher PASI 90 and PASI 100 rates at earlier time points. The 300 mg dose also provided a more durable response following Week 16. Thus, results from the study indicate that the highest dose (300 mg) provided the greatest efficacy.

Results from the study also suggest that additional dosing, if given during the induction period, might have further improved efficacy at Week 16. This suggestion is based on incremental benefits observed following a third dose administered to Week 16 non-responders when assessed within 4 week to 8 weeks of that dose. Model-based analyses and simulations indicate that 250 mg doses administered at Weeks 0, 4, 8, and 12 (1000 mg total) will maximize efficacy at the end of a 16-week induction period. A dosing regimen of 250 mg SC Q8W during the maintenance period is expected to maintain or further enhance the efficacy achieved at the end of the Induction Period. The 250 mg dose is expected to achieve exposures and efficacy that are not distinguishable from that observed with 300 mg dosing. A second maintenance dosing regimen of 125 mg Q8W SC may maintain efficacy on a lower dosing regimen. This second dosing regimen is expected to result in mirikizumab concentrations that have, in individual subjects, minimal overlap with the concentrations produced with the 250 mg mirikizumab Q8W SC regimen.

1-27. (canceled)

28. A method of treating psoriasis comprising administering mirikizumab to a patient, said method comprising:
a) administering four induction doses of mirikizumab to the patient by subcutaneous injection at 4 week intervals, wherein each induction dose comprises 250 mg of mirikizumab; and
b) administering maintenance doses of mirikizumab to the patient by subcutaneous injection at 8 week intervals, wherein the first maintenance dose is administered 4 weeks after the last induction dose is administered and wherein each maintenance dose comprises 125 mg or 250 mg of mirikizumab,
wherein the psoriasis is moderate to severe plaque psoriasis.

29-71. (canceled)
